(19) United States
(12) Patent Application Publication
(10) Pub. No.: US 2025/0188425 A1
(43) Pub. Date: Jun. 12, 2025

(54) Additive Solution for Erythrocyte Concentrates

(71) Applicants: Blutspendedienst der Landesverbände des Deutschen Roten Kreuzes Niedersachsen, Sachsen-Anhalt,, Springe (DE); Blutspendedienst des Bayerischen Roten Kreuzes, gemeinnützige Gesellschaft mit beschränkter Haftung, München (DE)

(72) Inventors: Ute Gravemann, Hemmingen (DE); Axel Seltsam, Isernhagen (DE); Wiebke Handke, Springe (DE)

(73) Assignees: Blutspendedienst der Landesverbände des Deutschen Roten Kreuzes Niedersachsen, Sachsen-Anhalt,, Springe (DE); Blutspendedienst des Bayerischen Roten Kreuzes, gemeinnützige Gesellschaft mit beschränkter Haftung, München (DE)

(21) Appl. No.: 18/292,599

(22) PCT Filed: Jul. 22, 2022
(86) PCT No.: PCT/DE2022/100535

§ 371 Date: Jan. 26, 2024

(30) Foreign Application Priority Data
Jul. 27, 2021 (DE) 10 2021 119 408.3

(51) Int. Cl. C12N 5/00

(52) U.S. Cl. CPC C12N 5/568; C12N 5/526

(57) ABSTRACT

The invention relates to an additive solution for storing erythrocyte concentrates, to erythrocyte concentrates that are provided with the additive solution, and to a process for producing erythrocyte concentrates diluted with the additive solution, comprising the step of irradiation with UV light, and to the use of the additive solution for storing erythrocyte concentrates.

Subject matter of the invention are an additive solution for storing red blood cell (RBC) concentrates, RBC concentrates provided with the additive solution, and a method for manufacturing RBC concentrates diluted with the additive solution, comprising the step of irradiation with UV light in the range of 300 to 200 nm as well as the use of the additive solution or of the components of the additive solution for storing RBC concentrates.

### TECHNICAL FIELD

RBC concentrates are “units of stored blood” consisting of red blood cells (RBCs, erythrocytes). In Germany, RBC concentrates as marketable forms are those, which contain at least 40 g of hemoglobin per unit and have a hematocrit of 0.5 to 0.7. The hematocrit (abbreviation: hct) refers to the proportion of the RBCs in the volume of the blood and is, in each case, defined hereinafter as the unit L/L. According to the prior art, RBC concentrates are usually diluted with an additive solution and are thus made more suitable for storage. The additive solution, often also referred to as storage solution, serves the purpose of suspending and storing the erythrocytes.

RBC concentrates can be manufactured in different ways. Manufacturing them from whole blood donations or using the apheresis procedure is common. In the apheresis procedure, RBCs are separated from the donor's blood in a dialysis-like apparatus using the continuous flow process, and the remaining blood components are guided back into the donor's circulatory system. For example, the anticoagulant ACD-A is used for mechanical blood donation.

Whole blood donations take place in that a certain volume of whole blood from a respective donor is filled into a respective blood bag made of a plastic material, e.g., 450 mL of whole blood by means of venous blood collection. The blood bag contains a stabilizer solution or the stabilizer solution is added to the whole blood in order to increase the shelf life of the whole blood and to counteract the clotting thereof.

A CPD stabilizer solution comprising citrate buffer, sodium dihydrogen phosphate and D-glucose is a typical stabilizer solution. As a rule, 63 mL of CPD stabilizer solution are used here for 450 mL of whole blood or 70 mL of CPD stabilizer solution for 500 mL of whole blood, respectively. The pH value of the whole blood is stabilized to 7.1 to 7.2 by means of the stabilizer solution.

When separating the whole blood donation into its individual components, the RBC concentrates are obtained, among others.

As a rule, a leukocyte depletion is performed on the whole blood or the RBC concentrates are subjected to a leukocyte depletion prior to the production of the RBC concentrates. Leukocyte depletion is understood to be the substantial removal of the donor leukocytes. This is a legal requirement in many countries, such as, e.g., in Germany. The leukocyte depletion takes place, e.g., in that the blood, after addition of the stabilizer solution or the RBC concentrate with the additive solution passes through a filter, which holds back the leukocytes or, e.g., during the apheresis, during which the leukocytes are separated from the RBCs by means of centrifugation. The used filters often consist of polyester fibers, which are compressed into packs, so that pores of a defined size are created, or of a polyurethane sponge with a defined pore size.

The filtration for the leukocyte depletion can take place directly after obtaining the blood and before obtaining the RBC concentrates or after storage of the RBC concentrates, optionally also at the patient's bedside first, prior to administering to a patient. It is common practice at least in Germany to perform the leukocyte depletion prior to storing the RBC concentrates.

To produce the RBC concentrates, the whole blood is centrifuged. The supernatant, which contains the plasma and the so-called buffy coat, consisting of thrombocytes and leukocytes, is separated and the erythrocytes remaining as centrifugate or sediment are suspended in an additive solution.

The RBC concentrate is optionally suspended once again beforehand in a nutrient solution, which can also be different from the additive solution, and centrifuged again in order to separate it again, so that remainders of the donor's blood plasma are replaced with the nutrient solution before the RBCs are suspended and stored in the additive solution. These washed RBC concentrates are useful in the case of insensitivities to prior transfusions (e.g., allergic reactions against proteins of the donor's plasma, e.g., in patients with IgA deficiency).

In order to adjust the RBC concentrates, as they can be obtained from the centrifugation, to a physiologically compatible viscosity, an additive solution has to be added, with which substances necessary for increasing the shelf life are supplied as well. The additive solution improves the survival rate and reduces the hemolysis of the erythrocytes, which occurs during the storage.

Additive solutions for RBC concentrates are known per se and have previously been proposed in different designs.

A solution is known from the U.S. Pat. No. 4,267,269, which, in addition to sodium chloride, glucose or fructose and adenine, contains the sugar alcohol mannite. A solution, which likewise contains sodium chloride, glucose or fructose and adenine, but sorbitol or xylite, respectively, as sugar alcohol, and optionally additionally guanosine, is described in the EP 0100419 A2. The EP 0301250 A1 discloses additive solutions, containing sodium chloride, disodium hydrogen phosphate and/or sodium hydrogen phosphate, glucose and/or fructose, sorbitol, mannite and/or xylite, adenine and/or guanosine and optionally colloids.

The additive solution SAG-M, e.g., has gained economic importance. It contains adenine, glucose, D-mannitol and sodium chloride (C. F. Högman, K. Hedlund, Y. Sahleström: “Red cell preservation in protein-poor media. III. Protection against in vitro hemolysis” in Vox Sang. 1981 November-December; 41 (5-6): 274-81).

PAGGS-mannitol (PAGGS-M), which is currently sold as follows as aqueous solution, is a further known additive solution:
47.4 mmol/L D-glucose monohydrate
8.0 mmol/L sodium dihydrogen phosphate dihydrate
8.0 mmol/L disodium hydrogen phosphate dihydrate
1.4 mmol/L adenine
1.4 mmol/L guanosine
54.9 mmol/L mannitol
72 mmol/L sodium chloride

In W. H. Walker, M. Netz, K. H. Gänshrit: “49 Tage Lagerung von Erythrozytenkonzentraten in Blutbeuteln mit der Konservierungslösung PAGGS-Mannitol”. Beitr. Infusionsther. 26 (1990): 55-59, Walter et al. have examined the shelf life of RBC concentrates with the additive solution PAGGS-M, wherein CPD was used as stabilizer solution.

It is known that the therapeutic application of blood preparations is associated with the risk that the recipients of the blood preparation get infected with viruses and/or bacteria. These include, for example, the viruses hepatitis B (HBV), West-Nile (WNV) and hepatitis C (HCV) as well as the AIDS viruses HIV-1 and HIV-2 or bacteria, such as, e.g., staphylococci or streptococci. The risk exists whenever no step for inactivating or eliminating, respectively, of the mentioned pathogens is applied during the production of the preparation.

A pathogen inactivation can be performed, e.g., by means of UV radiation. A method of this type is known, e.g., from the WO 2007/076832 A1. Ultraviolet (UV) light is differentiated, depending on wavelength. In terms of this application, the following definition is made: UVA: 400 to 320 nm, UVB: 320 to 280 nm and UVC: 280 to 200 nm. It is known that viruses as well as bacteria can be inactivated by irradiation with short-wave ultraviolet (UV) light, that is, in the wave range below approx. 320 nm (UVB and UVC), for instance in blood plasma or in cellular blood preparations. Above 320 nm, the energy of the radiation is too low to effectively inactivate microorganisms and viruses. In contrast to chemical, photochemical and photodynamic methods for pathogen inactivation, irradiation solely with UV light generally has the advantage of being effective on its own and of not requiring the addition of reactive chemicals or photoactive substances.

### OBJECT OF THE INVENTION

It is the object of the invention to improve the shelf life of RBC concentrates, in particular for RBCs, which were subjected to a UV irradiation before they are introduced into the additive solution, or also to provide a medium for the UV irradiation of the RBCs and the storage.

### SUMMARY OF THE INVENTION

The invention is characterized by the independent patent claims, preferred embodiments are subject matter of the subclaims and/or are described below.

In addition to water, the additive solution according to the invention comprises the following components:
12 to 50 mmol/L, in particular 17 to 50 mmol/L or 20 to 25 mmol/L, of disodium hydrogen phosphate (Na₂HPO₄);
0.1 to 3.5 mmol/L, in particular 1.5 to 2.5 mmol/L, of adenine;
10 to 90 mmol/L, in particular 45 to 55 mmol/L, of D-glucose;
0.1 to 3 mmol/L, in particular 1.25 to 1.75 mmol/L, of guanosine;
10 to 80 mmol/L, in particular 20 to 60 mmol/L or even 35 to 45 mmol/L, of sodium chloride; and
10 to 50 mmol/L, in particular 14 to 50 mmol/L or 25 to 35 mmol/L, of trisodium citrate.

The additive solution in particular consists of the following components:
12 to 50 mmol/L, in particular 17 to 50 mmol/L or 20 to 25 mmol/L, of disodium hydrogen phosphate (Na₂HPO₄);
0.1 to 3.5 mmol/L, in particular 1.5 to 2.5 mmol/L, of adenine;
10 to 90 mmol/L, in particular 45 to 55 mmol/L, of D-glucose;
0.1 to 3 mmol/L, in particular 1.25 to 1.75 mmol/L, of guanosine;
10 to 80 mmol/L, in particular 20 to 60 mmol/L or 35 to 45 mmol/L, of sodium chloride;
10 to 50 mmol/L, in particular 14 to 50 mmol/L or 25 to 35 mmol/L, of trisodium citrate;
wherein the remainder is water.

The above components can in each case also be used as their hydrates. The components are generally present to be dissociated in solution.

Concentrates or dilutions, in particular dilutions of the above additive solutions, are likewise claimed.

The additive solution preferably has a pH of greater than 7, preferably greater than 7.5, in particular a pH of 8 to 9, in each case at 22° C.

The osmolality of the additive solution is preferably from 260 to 300 mOsm/kg. The measurement of the osmolality takes place by means of freezing point depression method (osmometer).

A method for producing RBC concentrates diluted with the additive solution, comprising the step of irradiation of the RBC concentrates and/or of starting materials of the RBC concentrates, such as whole blood, with UV light in the range of 300 to 200 nm, in particular 280 to 220 nm, and preferably 260 to 240 nm (hereinafter generally also referred to as “UV irradiation” or “UV-irradiated”), is furthermore subject matter of the invention.

In the following three designs, the method for producing RBC concentrates comprises the following steps:
irradiation of whole blood or diluted whole blood with UV radiation, obtaining an RBC concentrate from the whole blood irradiated in this way by adding the additive solution or the components of the additive solution;
   or
irradiation of a RBC concentrate, comprising the additive solution or the components of the additive solution, under UV irradiation, wherein the RBC concentrate is preferably a diluted RBC concentrate with an hct of less than 0.5 and is concentrated to an hct of greater than or equal to 0.5 after the UV irradiation;
   or
irradiation of a diluted RBC concentrate, comprising a second additive solution under UV irradiation, wherein the diluted RBC concentrate preferably has an hct of less than 0.5 and is concentrated to an hct of greater than 0.5 after the UV irradiation,
wherein the second additive solution is replaced with the additive solution or the components of the additive solution after the irradiation at least at greater than 75% by weight, based on the second additive solution, preferably replaced completely;
   wherein the UV irradiation in each case takes place at a wavelength of 300 to 200 nm, in particular 280 to 220 nm and preferably 260 to 240 nm. When the components of the additive solution are added, this means that they are added so that the same concentrations result in each case as if the additive solution had been added. In this respect, this is the same as if the additive solution had been added.

The invention thus furthermore relates to a diluted RBC concentrate with an hct of 0.1 to 0.4, preferably 0.25 to 0.35, containing the above additive solution, in particular a leukocyte-depleted diluted RBC concentrate. The diluted RBC concentrate is obtained, e.g., from a 1:2 dilution of a RBC concentrate with the additive solution. RBC concentrates with an hct of less than 0.5 are also referred to as “diluted RBC concentrates” herein.

The RBC concentrate is preferably made up as follows, comprising (L=liter):
erythrocytes 0.40-0.80 L/L, in particular 0.50-0.70 L/L
additive solution 0.10-0.60 L/L, in particular 0.25-0.50 L/L
and optionally
stabilizer solution 0.0001-0.10 L/L, in particular CPD stabilizer solution
human plasma 0.0001-0.2 L/L

The sum of the numbers in each case adds up to a numerical value of 1 or less than 1 L/L, in particular 1 L/L.

If the UV irradiation took place on the whole blood (WB), the composition of the RBC concentrates is in particular as follows, comprising or consisting of:
erythrocytes 0.5-0.7 L/L
additive solution 0.27-0.48 L/L
CPD stabilizer solution 0.03-0.09 L/L
human plasma 0.025-0.15 L/L or 0.015-0.025 L/L
or, after UV irradiation of the RBC concentrates, if the UV irradiation did not take place on the whole blood (WB), comprising or consisting of:
erythrocytes 0.5-0.7 L/L
additive solution 0.27-0.49 L/L
CPD stabilizer solution 0.001-0.009 L/L or 0.001-0.014 L/L
human plasma 0.005-0.05 L/L

According to one embodiment, a suitable CPD stabilizer solution is constructed as follows, containing:
trisodium citrate dihydrate 80 to 100 mmol/L in particular 89.4 mmol/L
citric acid monohydrate 13 to 18 mmol/L, in particular 15.6 mmol/L
NaH₂PO₄ dihydrate 13 to 19 mmol/L, in particular 16.1 mmol/L
D-glucose monohydrate 115 to 140 mmol/L, in particular 128.7 mmol/L

According to one embodiment, the CPD stabilizer solution is added to the whole blood in a volume ratio of 1:6.1 to 1:8.1, in particular 1:7.14 (in each case V/V), e.g., 63 mL of CPD stabilizer solution for 450 mL of whole blood or 70 mL of CPD stabilizer solution for 500 mL, namely before the additive solution is used. According to the reprocessing, the above components of the CPD stabilizer solution then remain in the RBC concentrate in certain concentrations.

Due to the fact that glucose and trisodium citrate are also contained in the stabilizer solution, the proportion thereof increases accordingly in the RBC concentrate if CPD stabilizer solution is used. It is also possible, however, to use other stabilizer solutions.

According to one embodiment, the RBC concentrate contains:
10 to 30 mmol/L, in particular 14 to 26 mmol/L, of D-glucose;
5 to 12 mmol/L, in particular 6 to 10 mmol/L, of disodium hydrogen phosphate, e.g., as dihydrate;
0.3 to 1.2 mmol/L, in particular 0.5 to 0.8 mmol/L, of adenine
0.25 to 0.9 mmol/L, in particular 0.4 to 0.7 mmol/L, of guanosine;
8 to 25 mmol/L, in particular 11 to 20 mmol/L, of sodium chloride;
6 to 18 mmol/L, in particular 8 to 16 mmol/L, of trisodium citrate;
and optionally
0.01 to 1 mmol/L, in particular 0.02 to 0.8 mmol/L, of sodium dihydrogen phosphate, e.g., as dihydrate;
0.01 to 1 mmol/L, in particular 0.02 to 0.8 mmol/L, of citric acid, e.g., as monohydrate.

This embodiment can be obtained, e.g., if the RBC concentrate was obtained by adding CPD or CPDA-1 stabilizer solution for the blood donation and additive solution according to the invention, wherein the sodium dihydrogen phosphate and the citric acid are then introduced by means of the CPD stabilizer solution.

The invention thus furthermore relates to the use of the additive solution or of the components of the additive solution for storing RBC concentrates.

The difference in the concentration of the CPD stabilizer solution and of the human plasma results from dilution of the EC with the additive solution and subsequent concentration. During the concentration, a portion of the supernatant is removed and thus also a portion of the originally contained plasma and of the CPD stabilizer solution.

### DETAILED DESCRIPTION OF THE INVENTION

According to one embodiment, RBC concentrates are isolated from individual blood donations or are obtained from individual donors by means of mechanical apheresis. The volume of the preparations generally lies between approx. 200 and 350 mL. The volume of whole blood donations mostly range between 400 and 500 mL. The preparations are each stored in flat plastic bags, generally at approx. 4° C. to 37° C. for whole blood and 4° C.+/−2° C. for RBC concentrate.

The leukocyte depletion can be performed at the level of the whole blood, i.e., prior to the first centrifugation of the whole blood, or at the level where the RBCs obtained by centrifuging are suspended and diluted in the additive solution, i.e., at the level of the RBC concentrates. As a rule, the leukocyte depletion takes place by means of filtration. During obtaining the erythrocytes by means of apheresis, a separate leukocyte depletion is not necessary, the leukocyte depletion already takes place as part of the apheresis here.

According to one design, the leukocyte depletion is performed on a RBC concentrate diluted with the additive solution according to the invention, with an hct of, e.g., 0.1 to 0.4. A UV irradiation then optionally takes place after the leukocyte depletion, and then a concentration to an hct of in particular 0.5 to 0.7. The leukocyte depletion can take place prior to or after UV irradiation, preferably before.

The UV irradiation can take place on the whole blood (WB), i.e., prior to the production of the RBC concentrate, or on the RBC concentrate. A UV-treated, pathogen-depleted RBC concentrate and optionally leukocyte-depleted RBC concentrate is obtained as product in the additive solution according to the invention.

Starting material for the UV irradiation of RBC concentrates are, e.g., RBC concentrates with an hct between 0.8 to 1, in particular between 0.8 and 0.98. According to one embodiment, they are adjusted to an hct of 0.5 to 0.7 by means of the additive solution according to the invention and are irradiated at this concentration with UV or preferably during further dilution (diluted RBC concentrate). In terms of the present application, a RBC concentrate with an hct of less than 0.5 is referred to as diluted RBC concentrate.

The leukocyte-depleted RBC concentrate can also be adjusted to an hct of 0.1 to 0.4, preferably 0.25 to 0.35, (diluted RBC concentrate) by means of further dilution with the additive solution, e.g., from a 1:2 dilution of the RBC concentrate with additive solution. The RBC concentrates diluted in this way are transferred into an irradiation bag via a sterile hose connection and are irradiated with UV light under vigorous movement.

The irradiated RBC concentrate is transferred into an empty bag and is concentrated to an hct of 0.5-0.8, in particular 0.5-0.7, e.g., by means of centrifugation and pressing out the supernatant.

It is known that pathogens can be inactivated in blood products by irradiation with short-wave ultraviolet (UV) light. This takes place according to the method of the invention in that the blood products are subjected to an irradiation with ultraviolet (UV) light at wavelengths of 300 to 200 (range UVB to UVC), in particular to an irradiation at wavelengths in the UVC range of 280 nm to 220 nm, in particular 260 to 240 nm.

The radiation energy is preferably from 0.3 to 10 J/cm², more preferably 2.5 to 5.5 J/cm² and particularly preferably 3 to 5 J/cm² for whole blood and with erythrocyte concentrates of preferably 1.5 to 4.5 J/cm² and particularly preferably 2 to 4 J/cm² (in each case based on the radiation energy acting on the blood product). The radiation energy acting on the container, such as an irradiation bag, is in fact greater because the irradiation bag typically absorbs radiation energy of the relevant wavelength, depending on the material.

If the irradiation takes place through a medium, which absorbs the UV radiation, the radiation energy is to be increased accordingly. Irradiation bags made of EVA (ethylene vinyl acetate) absorb, e.g., approximately 30 to 40% of the radiation energy. The irradiation in particular takes place at a temperature of the blood product of 2 to 37° C.

A method of this type for the UV irradiation is known, e.g., from the WO 2007/076832 A1 and is applied to the RBC concentrates according to the present invention. According to this, the preparations, i.e., donor blood (whole blood) and/or RBC concentrates are moved in a suitable manner in an irradiation bag, so that a constant circulation of the samples takes place in the container. The movement thereby takes place so vigorously that layers, which are so thin that they can be penetrated by the used light, are formed in some areas within the liquid or suspension, respectively. The movement takes place so that liquid or suspension, respectively, is mixed effectively in the bag. Both is realized in particular if, for example, the following conditions are at hand:
1. The irradiation bags are flexible.
2. The irradiation bags are filled to maximally 40%, in particular maximally 30%, in particular maximally 15%, of the maximum filling volume.
3. The bags are moved vigorously, e.g., either horizontally (linearly in back and forth direction or in a circular or elliptical shape) and/or vertically (rocked).

In connection with the constant mixing, which takes place simultaneously, the entire preparation (and the pathogens contained therein) is ultimately irradiated and it is thus pathogen-reduced.

The irradiation bags can be shaken by means of an orbital shaker, platform shaker, rocking shaker or wobble shaker and are preferably moved for at least three quarters of the entire irradiation time. The irradiation bags typically have a volume of up to 5000 mL. If the irradiation bags are placed on one side, the height of the irradiation bag changes constantly during and due to the movement or shaking, respectively, over the entire upper surface of the irradiation bag, which is in contact with the bag content, based on the distance along the surface normal between surface, on which the irradiation bag rests, and point of intersection with the upper surface of the irradiation bag.

The irradiation bags are made of UV-transparent plastic material. Suitable plastics are, e.g., ethylene vinyl acetate and polyolefins with film thicknesses of, e.g., 1 mm and less, in particular film thicknesses of less than 0.5 mm. The irradiation bags are formed to be flat and preferably do not have any absorption maxima in the range from 200 to 320 nm. In the horizontal filled state, the irradiation bags have a thickness of only a few mm, e.g., less than 10 mm and in particular 5 mm, preferably even less than 3 mm and are intended to receive sample volumes of, e.g., up to 600 mL. The maximum capacity (volume) of the irradiation bag, however, is greater than the actual sample volume contained therein, preferably by at least a factor of 3, generally by at least a factor of 5 or at least 10. For example, the irradiation bag when horizontal has a base surface of 19×38 cm and a filling volume of 500 to 600 mL, thus resulting in an average filling height of 6.9 to 8.3 mm in the horizontal and rest state.

Each UV-irradiated unit can preferably be traced back to a donor.

Hematocrit (hct) identifies the proportion of the cellular components in the blood. Normal hct values in the blood are between 0.42 and 0.5 in men and between 0.37 and 0.45 in women. In the present case, it is specified in L/L. Due to the fact that the erythrocytes physiologically represent 99% of the total volume of the blood cells, the hct value corresponds approximately to the proportion of the cell volume.

The hct is determined by centrifuging a non-clotting blood sample in a tube according to DIN 58933-1:1995-01. The coagulation of the blood is thereby prevented by adding anticoagulants, such as EDTA (ethylenediamine tetraacetate) or heparin. The heavier RBCs separate from the plasma, the height of the erythrocyte column is measured in relation to the total blood column. The boundaries between erythrocytes, leukocytes/thrombocytes and blood plasma can be seen with the naked eye. If the leukocytes and/or thrombocytes and blood plasma are already separated, the solid body, which settles, consists almost exclusively of erythrocytes.

### Experimental Part

Influence of the additive solution UG65 on the quality of the UVC-irradiated blood preparation as well as effectiveness of the pathogen inactivation of blood preparations by means of UV irradiation.

### Production of the Components

Whole blood donations (450-500 mL) were collected in 70 mL of the anticoagulant CPD (day 0), hereinafter collectively referred to as whole blood donation, and were stored overnight at room temperature. In addition to water, the anticoagulant CPD contained the following components:

| | mmol/L |
|---|---|
| trisodium citrate dihydrate | 89.4 |
| citric acid monohydrate | 15.6 |
| NaH₂PO₄ dihydrate | 16.1 |
| D-glucose monohydrate | 128.7 |

On day 1, the whole blood was used directly for pathogen inactivation experiments or was further processed into the RBC concentrate. For this purpose, the erythrocytes were obtained after centrifugation in a conventional bag centrifuge and automatic component separation by means of a press-out machine as “dry” RBC concentrate and were subsequently suspended in the respective specified additive solution (110 mL). The depletion of the leukocytes took place by means of filtration at the level of the whole blood (“whole blood filtration”) or of the RBC concentrate (filtration after centrifugation and pressing out) by means of a leukocyte depletion filter.

Composition of the additive solution UG65:

The aqueous solution contains the following components:
22.7 mmol/L disodium hydrogen phosphate dihydrate
1.85 mmol/L adenine
51.6 mmol/L D-glucose monohydrate
1.44 mmol/L guanosine
40 mmol/L sodium chloride
28.4 mmol/L trisodium citrate dihydrate
Remainder water

Composition of the additive solution SAG-M:

The aqueous solution contains the following components:
1.25 mmol/L adenine
45.4 mmol/L D-glucose monohydrate
28.8 mmol/L D-mannitol
150 mmol/L sodium chloride
Remainder water

### Pathogen Inactivation by Means of UV Irradiation

Whole blood (520-570 mL) or RBC concentrate (600 mL, hct approx. 0.3) diluted in additive solution were filled into a UV-permeable bag (19×38 cm base surface made of EVA) and were irradiated on a UV irradiation system (Macotronic UV) with UVC light (254 nm) with a UVC dose of 4.5 J/cm² (EC) or 6 J/cm² (WB), respectively, and were shaken simultaneously (300 rpm).

A conventional RBC concentrate with an hct of 0.5 to 0.7 was subsequently obtained from the whole blood or the diluted RBC concentrate by means of centrifugation and automatic separation.

The information relating to the irradiated energy acts on the exterior of the irradiation bag. Approx. 50 to 75%, in the present case an estimated 60% of the irradiated energy penetrates the irradiation bag. The irradiation bag was irradiated from above and from below.

### Determination of the Quality Parameters

The hemolysis rate [%] is defined as the percentage of the free hemoglobin in the supernatant of the RBC concentrates compared to the total content.

Hemolysis (%)=((100−hematocrit*100)×free hemoglobin in the supernatant/total hemoglobin).

The hct was determined by means of hematocrit centrifuge (Haematokrit 210, Hettich). Free hemoglobin in the supernatant was determined photometrically by means of the 3-wavelength method according to Harboe (see: M. Harboe, A method for determination of hemoglobin in plasma by near-ultraviolet spectrophotometry. Scand J Clin Lab Invest, 1959. 11 (1): p. 66-70). Total hemoglobin was measured by means of hematology machine (XS1000i or XN550, Sysmex).

The determination of the glucose and lactate concentration took place by means of the blood gas analyzer ABL90 FLEX (radiometer). The ATP content of the erythrocytes was measured by means of the commercially available kit ATP Hexokinase FS (DiaSys Greiner). The pH was determined at 22° C. using a conventional pH meter. The volume was determined by means of weighing in consideration of the specific density of the RBC concentrate.

### Experiment 1

### Quality Parameters of RBC Concentrates UVC-Irradiated in UG65 and Stored in UG65

RBC concentrates (n=9) in additive solution UG65 were UVC-irradiated and concentrated again as described above. The finished RBC concentrates with an hct of approx. 0.6 were subsequently stored at 4±2° C. and samples for determining the in vitro quality were collected weekly.

### Results

The UVC-irradiated RBC concentrates had an hct between 0.59 and 0.64 and thus corresponded to the Guidelines of the Council of Europe. The hemolysis rate increased over the course of the storage. On day 36 of the storage, however, all nine RBC concentrates showed a hemolysis rate of below 0.8% and thus met the quality requirements of the Guideline des Council of Europe.

The pH value of the UVC-irradiated RBC concentrates was 7.14±0.06 on day 2 and decreased over the course of the storage to 6.54±0.05 on day 36. Parallel thereto, the glucose content of the RBC concentrates decreased from 37.8±1.6 to 23.7±1.9 and the lactate concentration increased from 6.9±0.6 to 30.4±1.6.

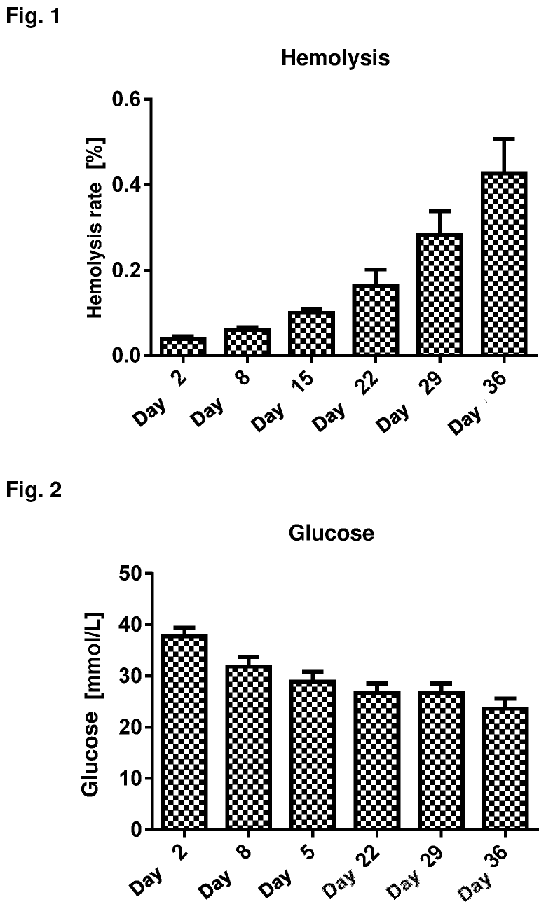

FIG. 1 shows the hemolysis rate over the course of the storage as a function of the time in days.

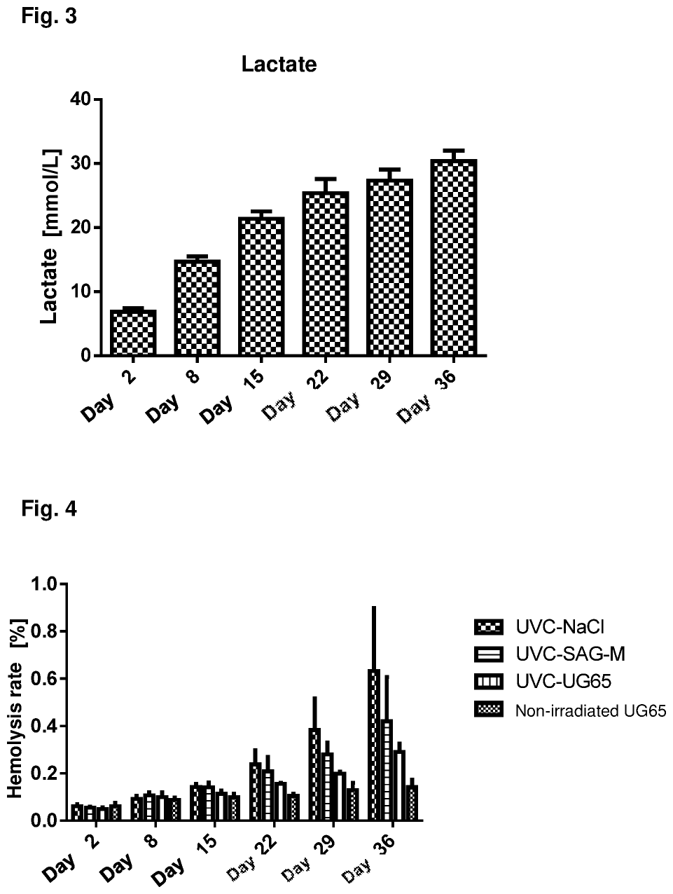

FIG. 2 shows the decrease of the glucose concentration of the RBC concentrates UVC-irradiated in UG65 and stored in UG65 and FIG. 3 shows the increase of the lactate concentration of the RBC concentrates UVC-irradiated in UG65 and stored in UG65 over the course of the storage.

The values are in each case specified as mean from 9 samples.

It was found that the newly developed additive is suitable for producing UVC-irradiated RBC concentrates in a good quality.

### Experiment 2

Influence of Different Additive Solutions During the UVC Irradiation with Subsequent Storage in the Additive Solution UG65

Four dry RBC concentrates were pooled and divided again. An RBC concentrate was suspended in 110 mL of UG65 and was filtered for the leukocyte depletion (untreated control without UVC irradiation).

For the three remaining RBC concentrates, a pooled RBC concentrate was in each case suspended in 110 mL of isotonic saline solution (NaCl 0.9%), with 110 mL of additive solution SAG-M and with 110 mL of additive solution UG65, filtered, and subsequently diluted to an hct of approx. 0.3 with the respective same additive solution. The UVC irradiation took place as specified above with a UVC dose of 4.5 J/cm². The UVC-irradiated RBC concentrates were centrifuged subsequently, the supernatant was removed and all of the erythrocytes were each suspended in additive solution UG65 again. The storage of the RBC concentrates in the additive solution UG65 took place at 4±2° C. and samples for determining the in vitro quality were collected weekly.

### Results

As shown in FIG. 4, the UVC-irradiation compared to the non-irradiated control led to an increased hemolysis rate. The hemolysis rate was highest when the RBCs were irradiated in the presence of NaCl or SAG-M. The best quality of the UVC-irradiated RBC concentrates was attained if the additive solution UG65 was already present during the irradiation.

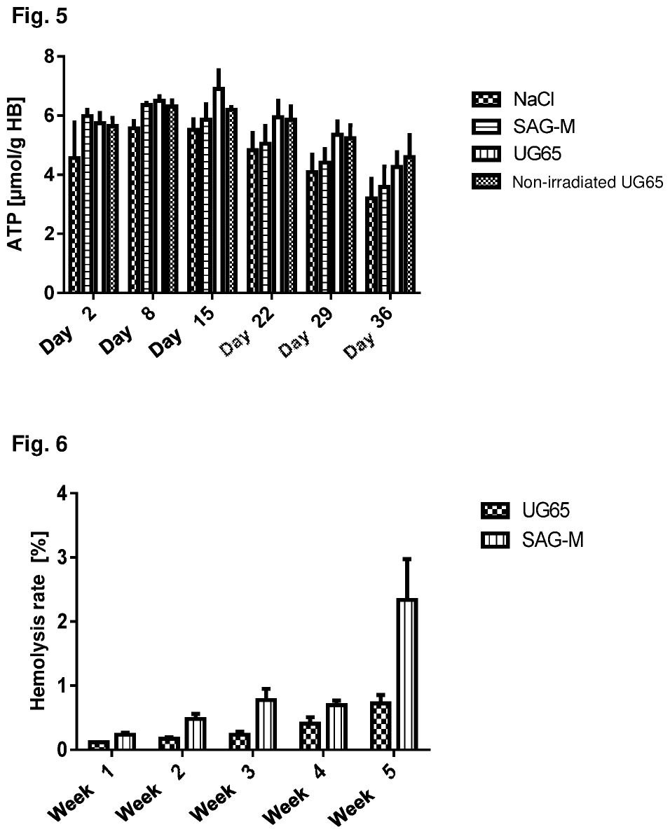

The ATP content as parameter for the energy status of the RBCs was likewise influenced by the UVC irradiation. The ATP content at the end of the storage was highest if the RBCs were irradiated in the presence of UG65 (FIG. 5). The irradiation of the RBC concentrates in the presence of NaCl or SAG-M led to a decrease of the ATP content compared to the non-irradiated control.

FIG. 4 specifies the hemolysis rate and FIG. 5 the ATP content of the UVC-irradiated RBC concentrates, which were irradiated in the presence of NaCl, SAG-M or UG65 and which were then stored in UG65. An EC, which was stored without UVC-irradiation in UG65, served as control.

It follows from the series of experiments that the dilution of the RBC concentrates with the additive solution UG65 during the UVC irradiation has a positive effect on the quality of the erythrocytes. The newly developed additive solution provides an advantage compared to other possible dilution solutions, such as saline solution or conventional additive solutions.

### Experiment 3

### Comparison of the Quality of UVC-Treated RBC Concentrates Irradiated and Stored in the Additive Solution UG65 Compared to RBC Concentrates Irradiated and Stored in the Conventional Additive Solution SAG-M

“Dry” RBC concentrate (hematocrit >0.8) was obtained as described above. Two dry RBC concentrates were pooled and split up again and were subsequently suspended once in 110 mL of the commercially available additive solution SAG-M (control) and once in 110 mL of the newly developed additive solution UG65 (test). The depletion of the leukocytes took place by means of filtration of these RBC concentrates using a conventional leukocyte depletion filter. After the filtration, the respective RBC concentrate was mixed with the same amount of the respective additive solution (w/w). 600 g of the diluted RBC concentrate were transferred into a UVC-permeable irradiation bag. The UVC irradiation took place as described above. A conventional RBC concentrate was subsequently obtained from the diluted RBC concentrate by means of centrifugation and automatic separation. The finished RBC concentrates (n=3, test and control) were stored at 4±2° C. and samples for determining the in vitro quality were collected weekly.

### Results

After production, the test and control RBC concentrates had comparable values for volume, hct and hemoglobin per unit (Table 1).

TABLE 1
Production data of UVC-irradiated RBC concentrates
(RBCC) in UG65 and SAG-M (n = 3)

| | Control (UVC-RBCC in | Test (UVC-RBCC in |
|---|---|---|
| | SAG-M) | UG65) |
| volume [mL] | 277 ± 12 | 285 ± 15 |
| hematocrit [L/L] | 0.55 ± 0.02 | 0.56 ± 0.02 |
| hemoglobin per unit | 51.8 ± 4.0 | 50.9 ± 4.6 |
| [g/unit] | | |

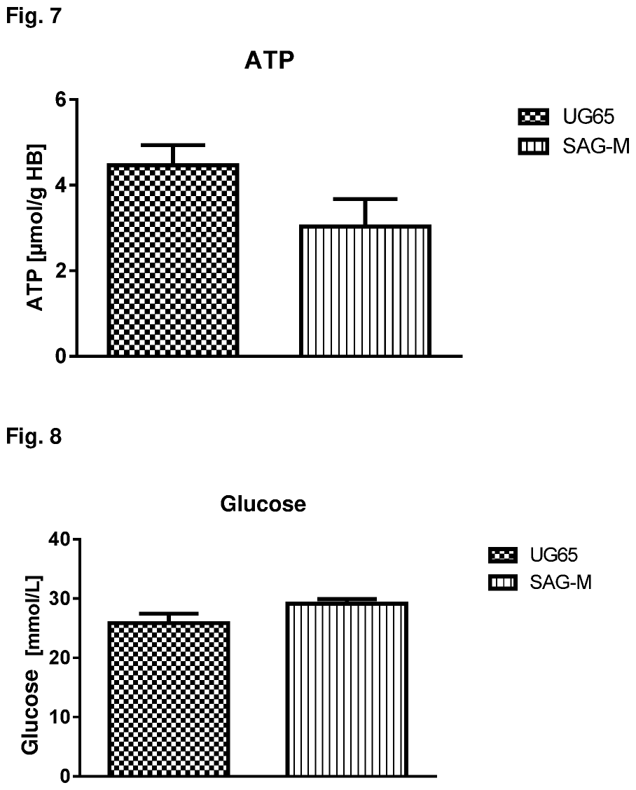

As most important quality parameter for RBC concentrates, the hemolysis rate of the RBC concentrates UVC-irradiated and stored in UG65 was significantly lower compared to the RBC concentrates UVC-irradiated and stored in conventional additive solution SAG-M (FIG. 6). In the course of the storage, all further quality parameters also showed significant differences between the control and test RBC concentrates (FIG. 7-9).

FIG. 6 shows the hemolysis rate in the course of the storage of RBCCs UVC-irradiated and stored in the same additive solution.

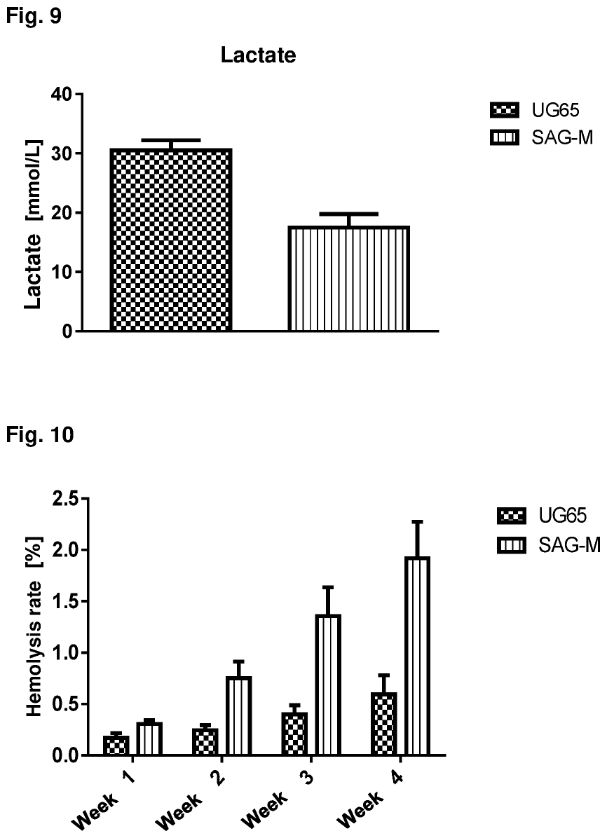

FIG. 7 shows the ATP content, FIG. 8 the glucose content and FIG. 9 the lactate content, in each case at the end of the storage (week 5) of RBCCs UVC-irradiated and stored with the same additive solution.

In the course of the storage, a significant advantage of the newly developed additive solution UG65 became apparent for the UVC irradiation of RBC concentrates. The RBC concentrates UVC-irradiated in UG65 and stored in UG65 were superior with regard to the quality of those in the conventional additive solution SAG-M.

### Experiment 4

Quality of RBC Concentrates after UVC Irradiation at the Level of the Whole Bloods, Comparison of the Additive Solution UG65 with Conventional Additive Solution SAG-M

Whole blood donations (approx. 570 mL) were irradiated with UVC as described above and were subsequently obtained by means of automatic component separation by means of a press-out machine as “dry” RBC concentrate (hematocrit >0.8). Two dry RBC concentrates were pooled and split up again and were subsequently suspended once in 110 mL of the commercially available additive solution SAG-M (control) and once in 110 mL of the newly developed solution UG65 (test). The depletion of the leukocytes took place by means of filtration of these RBC concentrates by means of a commercially available leukocyte depletion filter. The RBC concentrates (n=4, test and control) were subsequently stored at 4 ±2° C. and samples for determining the in vitro quality were collected weekly.

### Results

After production, the test and control RBC concentrates had comparable values for volume, hct and hemoglobin per unit (Table 2).

TABLE 2
Production data of RBC concentrates (RBCCs) produced from
UVC-irradiated whole blood stored in UG65 or SAG-M (n = 4)

| | Control | Test (UVC-RBCC in |
|---|---|---|
| | (UVC-RBCC in SAG-M) | UG65) |
| volume [mL] | 282 ± 12 | 284 ± 13 |
| hematocrit [L/L] | 0.56 ± 0.01 | 0.59 ± 0.02 |
| hemoglobin per unit | 53.8 ± 4.1 | 55.2 ± 4.0 |
| [g/unit] | | |

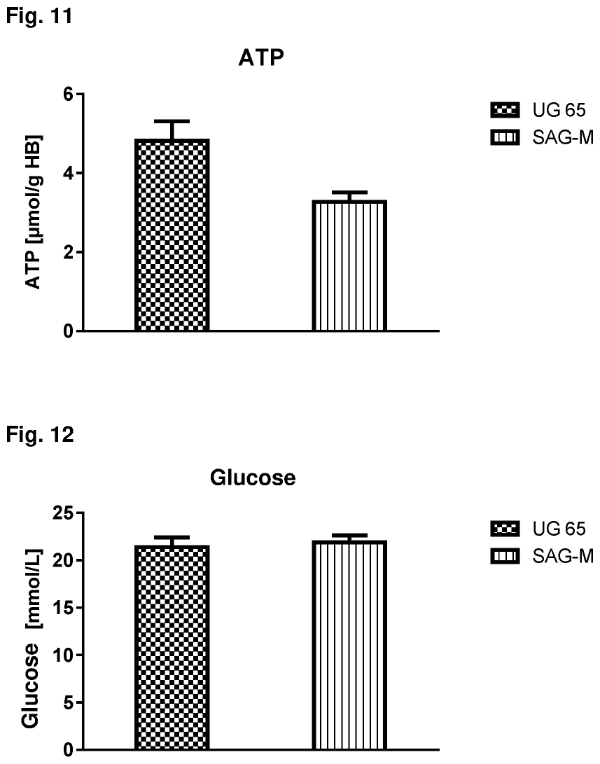

As most important quality parameter for RBC concentrates, the hemolysis rate of the RBC concentrates obtained from UVC-irradiated whole blood in UG65 was significantly lower compared to the RBC concentrates obtained from UVC-irradiated whole blood in the conventional additive solution SAG-M (FIG. 10). In the course of the storage, further quality parameters also showed significant differences between the control and test RBC concentrates (FIGS. 11-13).

FIG. 10 shows the hemolysis rate in the course of the storage of RBCCs obtained from UVC-irradiated whole blood, stored in UG65 and SAG-M.

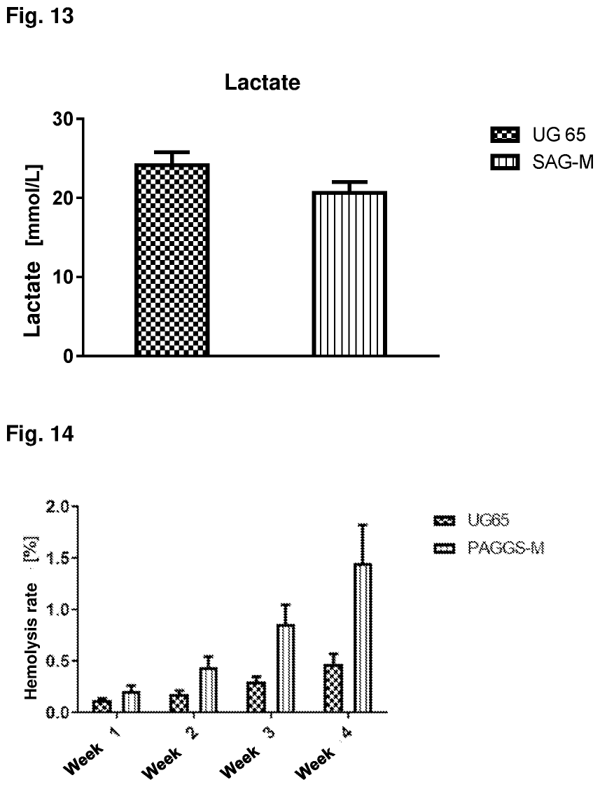

FIG. 11 shows the ATP content, FIG. 12 the glucose content and FIG. 13 the lactate content, in each case at the end of the storage (week 4).

In the course of the storage, a significant advantage of the newly developed additive solution UG65 became apparent for the production of RBC concentrates from UVC-irradiated whole blood. The quality of RBC concentrates from UVC-irradiated whole blood in UG65 is superior to those in the conventional additive solution SAG-M.

### Experiment 5

### Bacteria Inactivation

The bacterial strains Klebsiella pneumoniae (PEI-B-P-08-01), Serratia marcescens (PEI-B-P-56) and Pseudomonas fluorescens (PEI-B-P-77) were propagated in CASA bouillon, mixed with human serum albumin and were stored frozen until use (see U. Gravemann, et al., Bacterial inactivation of platelet concentrates with the THERAFLEX UV-Platelets pathogen inactivation system. Transfusion, 2019. 59 (4): p. 1324-1332). Whole blood or diluted RBC concentrates with an hct of approx. 0.3 were spiked with approx. 1×10⁶ KBE/mL bacterial suspension (n=3 for each used bacterium) and were subsequently irradiated with UVC light and by shaking. A conventional RBC concentrate was subsequently prepared from the diluted RBC concentrate or the whole blood. Samples were collected at different points in time and the bacteria titer was determined by plating on agar plates.

### Results

The bacteria were inactivated in RBC concentrates (Table 3) as well as in the whole blood (Table 4) in a dose-dependent manner with log-reduction factors between 4 and 6 log levels. The experiments prove the bacteria inactivation efficiency of the method.

TABLE 3
Bacteria inactivation in RBC concentrate (RBCC) (titer in KBE/mL,
m, n = 3), with additive solution UG65 according to the invention

| | Klebsiella | Serratia | Pseudomonas |
|---|---|---|---|
| | pneumoniae | marcescens | fluorescens |
| | (PEI-B-P-08-01) | (PEI-B-P-56) | (PEI-B-P-77) |
| m = mean | M | M | M |
| 0.0 J/cm² | 1.6E+06 | 2.4E+06 | 9.9E+05 |
| 4.5 J/cm² | 12.8 | 2.7 | ≤1.5 |
| RBCC after | 10.3 | 3.7 | ≤1.5 |
| reconcentrating | | | |

TABLE 4
Bacteria inactivation in whole blood (titer in KBE/mL, m,
n = 3), without additive solution during the UV irradiation

| | Klebsiella | Serratia | Pseudomonas |
|---|---|---|---|
| | pneumoniae | marcescens | fluorescens |
| | (PEI-B-P-08-01) | (PEI-B-P-56) | (PEI-B-P-77) |
| m = mean | m | m | M |
| 0.0 J/cm² | 1.2E+06 | 1.2E+06 | 1.2E+06 |
| 6.0 J/cm² | 125.3 | 2.4 | 10.2 |
| RBCC | 1.8 | 1.3 | 15.8 |

### Experiment 6

### Virus Inactivation

EMCV (strain EMC, ATCC VR129B), Sindbis virus (strain Ar339, ATCC VR-68) and VSV (strain Indiana, ATCC VR-158) were propagated and titrated on Vero cells (African Green monkey cell line from kidney tissue, ATCC, Bio Whittaker No. BE76-108B). The cultivation and titration took place as described in Mohr et al. (H. Mohr et al., A novel approach to pathogen reduction in platelet concentrates using short-wave ultraviolet light. Transfusion, 2009. 49 (12): p. 2612-24).

Whole blood or RBC concentrates diluted in the additive solution UG65 with an hct of approx. 0.3 were spiked with virus suspension (10% v/v, n=3 for each used virus) and were subsequently irradiated with UVC light while being shacked. A conventional RBC concentrate was subsequently prepared from the diluted RBC concentrate or the whole blood. Samples were collected at different points in time and the virus titer was determined by means of end point titration. Upon reaching the detection limit, the large volume plating was applied instead of the end point titration.

The viruses were inactivated in RBC concentrate (Table 5) as well as in the whole blood (Table 6) in a dose-dependent manner with log reduction factors between 3 and 5 log levels. The experiments prove the virus inactivation efficiency of the method.

TABLE 5
Virus inactivation in RBC concentrate (RBCC) (log₁₀TCID₅₀,
m, n = 3) with additive solution UG65 according to the invention

| | VSV | EMCV | Sindbis |
|---|---|---|---|
| | m | m | M |
| 0 J/cm² | 8.43 | 6.82 | 6.92 |
| 4.5 J/cm² | ≤3.54 | 3.68 | 2.62 |
| RBCC after | ≤3.54 | 3.76 | 2.65 |
| reconcentrating | | | |

TABLE 6
Virus inactivation in whole blood (log₁₀TCID₅₀, m, n =
3), without additive solution during the der UV irradiation

| | VSV | EMCV | Sindbis |
|---|---|---|---|
| | m | m | m |
| 0.0 J/cm² | 8.19 | 6.86 | 6.64 |
| 6.0 J/cm² | 2.64 | 3.82 | 3.58 |
| RBCC | 2.49 | 3.80 | 2.49 |

### Experiment 7

Quality of RBC Concentrates after UVC Irradiation at the Level of the Whole Bloods, Comparison of the Additive Solution UG65 with the Additive Solution PAGGS-M

Two whole blood donations (in each case approx. 500 ml of whole blood+70 mL of CPD stabilizer solution) were pooled and divided again. The whole blood units were irradiated with UVC as described above and were subsequently processed by means of automatic component separation by means of a press-out machine into “dry” RBC concentrates (hematocrit >0.8).

Two dry RBC concentrates were pooled and split up again and were subsequently suspended once in 110 mL of a commercially available additive solution PAGGS-M as described above (control, composition as described further above) and once in 110 mL of the newly developed solution UG65 (test). The depletion of the leukocytes took place by means of filtration of these RBC concentrates by means of a conventional leukocyte depletion filter. The RBC concentrates (n=4, test and control) were subsequently stored at 4±2° C. and samples for determining the in vitro quality were collected weekly. When using the same amount of CPD, the RBC concentrates differ in their composition at least in each case in that the RBC concentrates, additives added according to the invention, did not have a mannitol and the content of disodium hydrogen phosphate and trisodium citrate was significantly greater in the RBC concentrates according to the invention, compared with those, which, in addition to CPD, also contained PAGGS-M.

TABLE 7
Concentration in the RBCC with additive solution UG65

| | | |
|---|---|---|
| D-glucose | mmol/L | 18.6 |
| sodium dihydrogen phosphate dihydrate | mmol/L | 0.03 |
| disodium hydrogen phosphate | mmol/L | 8.1 |
| adenine | mmol/L | 0.7 |
| guanosine | mmol/L | 0.5 |
| sodium chloride | mmol/L | 14.2 |
| trisodium citrate | mmol/L | 10.3 |
| citric acid monohydrate | mmol/L | 0.03 |

TABLE 8
Concentration in the RBCC with additive solution PAGGS-M

| | | |
|---|---|---|
| D-glucose | mmol/L | 17.1 |
| sodium dihydrogen phosphate dihydrate | mmol/L | 2.9 |
| disodium hydrogen phosphate | mmol/L | 2.9 |
| adenine | mmol/L | 0.5 |
| guanosine | mmol/L | 0.5 |
| sodium chloride | mmol/L | 25.6 |
| trisodium citrate | mmol/L | 0.2 |
| citric acid monohydrate | mmol/L | 0.03 |
| mannitol | mmol/L | 19.5 |

### Results

After production, the test and control RBC concentrates had comparable values for volume, hct and hemoglobin per unit (Table 9).

TABLE 9
Production data from RBC concentrates (RBCCs) produced from
UVC-irradiated whole blood stored in UG65 or PAGGS-M (n = 4)

| | Control | |
|---|---|---|
| | (UVC-RBCC in | Test (UVC-RBCC in |
| | PAGGS-M) | UG65) |
| volume [mL] | 266 ± 17 | 268 ± 15 |
| hematocrit [L/L] | 0.57 ± 0.01 | 0.58 ± 0.00 |
| hemoglobin per unit | 56.1 ± 5.0 | 56.9 ± 4.8 |
| [g/unit] | | |

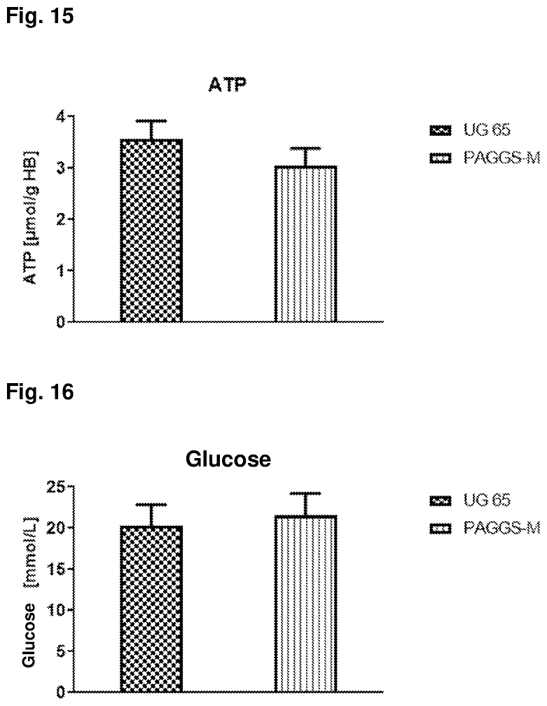

As the most important quality parameter for RBC concentrates, the hemolysis rate of the RBC concentrates prepared from UVC-irradiated whole blood in UG65 was significantly lower compared to the RBC concentrates prepared from UVC-irradiated whole blood in the conventional additive solution PAGGS-M (FIG. 14). In the course of the storage, further quality parameters also showed significant differences between the control and test RBC concentrates (FIGS. 15 to 17).

FIG. 14 shows the hemolysis rate in the course of the storage of RBCCs prepared from UVC-irradiated whole blood, stored in UG65 and PAGGS-M.

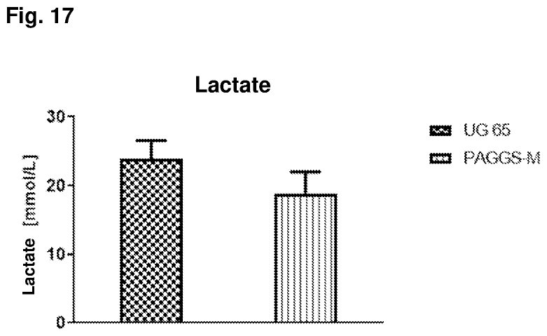

FIG. 15 shows the ATP content, FIG. 16 the glucose content and FIG. 17 the lactate content, in each case at the end of the storage (week 4).

In the course of the storage, a significant advantage of the newly developed additive solution UG65 became apparent for the manufacture of RBC concentrates from UVC-irradiated whole blood. The quality of RBC concentrates from UVC-irradiated whole blood in UG65 is superior to those in the additive solution PAGGS-M.

What is claimed is:

1. A method for production of red blood cell (RBC) concentrates, wherein the RBC concentrate comprises erythrocytes, which were UV-irradiated, and the method comprises the following steps:
irradiation of whole blood or diluted whole blood with UV radiation, obtaining an RBC concentrate from the whole blood so irradiated by adding an additive solution or components;
or
irradiation of a RBC concentrate or of a diluted RBC concentrate with an hct of less than 0.5, which is concentrated to an hct of greater than or equal to 0.5 after the UV irradiation, each comprising an additive solution or components;
or
irradiation of a diluted RBC concentrate, comprising a second additive solution, wherein the second additive solution is at least partially replaced with an additive solution or components after the irradiation at least at greater than 75% by weight, based on the second additive solution;
wherein the UV irradiation in each case is conducted at a wavelength of 300 to 200 nm; wherein according to an alternative A
the additive solution is an additive solution comprising along with water at least the following components:
12 to 50 mmol/L of disodium hydrogen phosphate;
18.01 to 3.5 mmol/L of adenine;
10 to 90 mmol/L of D-glucose;
18.01 to 3 mmol/L of guanosine;
10 to 80 mmol/L of sodium chloride; and
10 to 50 mmol/L of trisodium citrate,
or according to an alternative B
the components are the following components of the additive solution:
disodium hydrogen phosphate; adenine; D-glucose; guanosine; sodium chloride; and
trisodium citrate,
and the components according to alternative B are used in such a quantity in order to obtain, as a result, the erythrocyte concentrate comprising
10 to 30 mmol/L of D-glucose;
5 to 12 mmol/L of disodium hydrogen phosphate;
0.3 to 1.2 mmol/L of adenine;
0.25 to 0.9 mmol/L of guanosine;
8 to 25 mmol/L of sodium chloride;
6 to 18 mmol/L of trisodium citrate.

2. The method according to claim 1, wherein the concentration in the additive solution of disodium hydrogen phosphate, adenine, D-glucose, guanosine, sodium chloride and/or trisodium citrate is, each individually or jointly, the following:
17 to 50 mmol/L, in particular 20 to 25 mmol/L, of disodium hydrogen phosphate;
1.5 to 2.5 mmol/L of adenine;
45 to 55 mmol/L of D-glucose;
1.25 to 1.75 mmol/L of guanosine;
20 to 60 mmol/L of sodium chloride, in particular 35 to 45 mmol/L of sodium chloride;
14 to 50 mmol/L, in particular 25 to 35 mmol/L, of trisodium citrate.

3. The method according to claim 1, wherein the additive solution consists of the specified components in the specified concentrations, each with the remainder being water.

4. The method according to claim 1, wherein the additive solution has a pH of greater than 7, preferably greater than 7.5, in particular a pH of 8 to 9, each at 22° C.

5. The method according to claim 1, wherein the osmolality of the additive solution is 260 to 300 mOsm/kg.

6. The method according to claim 1, wherein the additive solution comprises
no mannitol or
no sorbitol or
no mannitol and no sorbitol.

7. The method according to claim 1, wherein the RBC concentrate comprises:
0.40 to 0.80 L/L, preferably 0.50 to 0.70 L/L, of erythrocytes and
0.10 to 0.60 L/L of additive solution, preferably 0.25 to 0.50 L/L, of additive solution,
wherein the sum of the proportions by volume in L/L in each case adds up to a numerical value of 1 or less than 1 L/L.

8. The method according to claim 1, wherein the RBC concentrate furthermore comprises:
0.0001-0.1 L/L of stabilizer solution, in particular CPD stabilizer solution, and/or
0.0001 to 0.2 L/L of human plasma.

9. The method according to claim 1, wherein the RBC concentrate comprises erythrocytes, which were UV-irradiated at the whole blood stage.

10. The method according to at least claim 1, wherein the RBC concentrate comprises erythrocytes, which were irradiated with UV radiation at the stage of a diluted RBC concentrate, and the diluted RBC concentrate preferably has an hct of less than 0.5 and is concentrated after the UV irradiation.

11. The method according to claim 1, wherein the RBC concentrate has an hct of 0.4 to 0.8, in particular 0.5 to 0.7.

12. The method according to claim 1, wherein the RBC concentrate comprises
no mannitol or
no sorbitol or
no mannitol and no sorbitol.

13. The method according to claim 1, wherein the RBC concentrate comprises according to alternative A:
10 to 30 mmol/L, in particular 14 to 26 mmol/L, of D-glucose;
5 to 12 mmol/L, in particular 6 to 10 mmol/L, of disodium hydrogen phosphate;
0.3 to 1.2 mmol/L, in particular 0.5 to 0.8 mmol/L, of adenine;
0.25 to 0.9 mmol/L, in particular 0.4 to 0.7 mmol/L, of guanosine;
8 to 25 mmol/L, in particular 11 to 20 mmol/L, of sodium chloride;
6 to 18 mmol/L, in particular 8 to 16 mmol/L, of trisodium citrate;
and optionally
0.01 to 1 mmol/L, in particular 0.02 to 0.8 mmol/L, of sodium dihydrogen phosphate; and
0.01 to 1 mmol/L, in particular 0.02 to 0.8 mmol/L, of citric acid;
or according to alternative B:
14 to 26 mmol/L of D-glucose;
6 to 10 mmol/L of disodium hydrogen phosphate;
0.5 to 0.8 mmol/L of adenine;
0.4 to 0.7 mmol/L of guanosine;
11 to 20 mmol/L of sodium chloride;
8 to 16 mmol/L of trisodium citrate;
and optionally
0.01 to 1 mmol/L, in particular 0.02 to 0.8 mmol/L, of sodium dihydrogen phosphate; and
0.01 to 1 mmol/L, in particular 0.02 to 0.8 mmol/L, of citric acid.

14. The method according to claim 1, wherein the RBC concentrate has less than 0.6 mmol/L, in particular less than 0.5 mmol/L, of sodium dihydrogen phosphate.

15. The method according to claim 1, wherein the method comprises the following steps:
irradiation of an RBC concentrate, comprising the additive solution or the components, wherein RBC concentrate is a diluted RBC concentrate with an hct of less than 0.5 and is concentrated to an hct of greater than or equal to 0.5 after the UV irradiation;
or
irradiation of a diluted RBC concentrate, comprising a second additive solution, wherein the diluted RBC concentrate has an hct of less than 0.5 and is concentrated to an hct of greater than 0.5 after the UV irradiation, wherein the second additive solution is at least partially replaced with the additive solution or the components after the irradiation at least at greater than 75% weight, based on the second additive solution, preferably replaced essentially completely.

16. The method according to claim 1, wherein the UV irradiation in each case is conducted at a wavelength of 280 to 220 nm and preferably 260 to 240 nm.

17. The method according to claim 1, wherein the additive solution or the components is/are added to a RBC concentrate with an hct of greater than or equal to 0.5.

18. An additive solution for storing red blood cell (RBC) concentrates, wherein the RBC concentrates comprises erythrocytes, which were UV-irradiated, wherein the additive solution comprises along with water at least the following components:
12 to 50 mmol/L of disodium hydrogen phosphate;
0.1 to 3.5 mmol/L of adenine;
10 to 90 mmol/L of D-glucose;
0.1 to 3 mmol/L of guanosine;
10 to 80 mmol/L of sodium chloride; and
10 to 50 mmol/L of trisodium citrate.

19. The additive according to claim 18, wherein the concentration of disodium hydrogen phosphate, adenine, D-glucose, guanosine, sodium chloride and/or trisodium citrate is, each individually or jointly, the following:
17 to 50 mmol/L, in particular 20 to 25 mmol/L, of disodium hydrogen phosphate;
1.5 to 2.5 mmol/L of adenine;
45 to 55 mmol/L of D-glucose;
1.25 to 1.75 mmol/L of guanosine;
20 to 60 mmol/L of sodium chloride, in particular 35 to 45 mmol/L of sodium chloride;
14 to 50 mmol/L, in particular 25 to 35 mmol/L, of trisodium citrate.

20. The additive according to claim 18, wherein the additive solution consists of the specified components in the specified concentrations, each with the remainder being water.

21. The additive according to claim 18, wherein the additive solution has a pH of greater than 7, preferably greater than 7.5, in particular a pH of 8 to 9, each at 22° C.

22. The additive according to claim 18, wherein the osmolality of the additive solution is 260 to 300 mOsm/kg.

23. The additive according to claim 18, wherein the additive solution comprises
no mannitol,
no sorbitol or
no mannitol and no sorbitol.

24. The additive according to claim 18, wherein the additive solution is a dilution of the additive solution with water or a concentrate of the additive solution.
